(19) United States
(12) Patent Application Publication
(10) Pub. No.: US 2023/0371549 A1
(43) Pub. Date: Nov. 23, 2023

(54) Pet Food Compositions

(71) Applicant: Hill's Pet Nutrition, Inc., Topeka, KS (US)

(72) Inventors: Dayakar BADRI, Lawrence, KS (US); Kiran PANICKAR, Lawrence, KS (US)

(73) Assignee: Hill's Pet Nutrition, Inc., Topeka, KS (US)

(21) Appl. No.: 18/321,970

(22) Filed: May 23, 2023

Related U.S. Application Data
(60) Provisional application No. 63/344,994, filed on May 23, 2022.

(51) Int. Cl. A23K 10/30; A23K 20/121; A23K 20/137

(52) U.S. Cl. CPC A23K 10/30; A23K 20/121; A23K 20/137

(57) ABSTRACT

Pet food compositions, and particularly, pet food compositions comprising a botanical component are disclosed herein. In accordance with a first aspect, provided is a pet food composition that includes an effective amount of a plurality of botanical ingredients selected from cinnamon, green tea, pomegranate, ginger, lime peel, tulsi, and a combination of two or more thereof.

### CROSS-REFERENCE TO RELATED PATENT APPLICATIONS

This application claims priority to U.S. Appl. No. 63/344,994, entitled “PET FOOD COMPOSITIONS,” filed on May 23, 2022, the content of which is incorporated herein by reference in its entirety for all purposes.

### BACKGROUND

Atopic dermatitis (AD) is a common type of chronic inflammatory skin disease found in pets. Canines particularly suffer from atopic dermatitis as one of the primary reasons for a veterinary visit. Typically, atopic dermatitis is characterized by pruritus (itchiness), erythema (redness), skin and ear secretions and alopecia (hair loss). Thus, atopic dermatitis is considered to significantly reduce the quality of life for pets suffering from atopic dermatitis.

In addition to a reduced quality of life, pets suffering from atopic dermatitis often suffer from pruritic skin disorder and/or skin membrane barrier dysfunction. Skin barrier plays a critical role in preventing the entry of allergens and microorganisms into the body. The physical skin barrier is localized in the uppermost area of the epidermis called stratum corneum. Further, the epidermis is continuously regenerated by terminally differentiating keratinocytes, a process called keratinization or cornification. Cornification begins with the migration of keratinocytes from the basal to upper layers during which keratinocytes also produce lipids and extrude them into the extracellular space to form extra-cellular enriched layers to maintain skin membrane barrier function. In addition, the presence of cytokines within the skin influences various processes of keratinocyte proliferation, differentiation and cornification.

It is believed that a combination of genetic, environmental, and immunological factors influence the pathogenesis of atopic dermatitis as well as the extent of the harm from such atopic dermatitis.

There is a present need for treatments, compositions, and methods for alleviating pets suffering from atopic dermatitis and the effects thereof.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to pet food compositions and, particularly, pet food compositions comprising a botanical component. In accordance with a first aspect, provided is a pet food composition that includes an effective amount of a plurality of botanical ingredients selected from cinnamon, green tea, pomegranate, ginger, lime peel, tulsi, and a combination of two or more thereof.

According to another aspect of the invention, provided is a pet food composition that includes a fat; a protein; and a botanical component. The botanical component typically comprises three to six botanical ingredients selected from about 0.05 to about 2 wt. % of cinnamon; from about 0.05 to about 2 wt. % of green tea; from about 0.05 to about 2 wt. % of pomegranate; from about 0.005 to about 1 wt. % of ginger; from about 0.2 to about 3 wt. % of lime peel extract; and from about 0.0006 to about 1 wt. % of tulsi.

In accordance with a further aspect of the invention, a method is provided for pruritus, erythema, alopecia, or skin and ear secretion in atopic dermatitis canine, the method comprising administering a pet food composition disclosed herein. The method may include providing and/or feeding the canine a pet food composition disclosed herein. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph showing the improvement in erythema, pruritus, and alopecia exhibited by canines consuming a control pet food composition, a first non-limiting, exemplary pet food composition, or a second non-limiting, exemplary pet food composition according to aspects of the invention;

FIGS. 2A-2C are bar graphs showing the improvement in pruritus, erythema, skin and ear secretions exhibited by canines consuming the control pet food composition or the second exemplary pet food composition in accordance with aspects of the invention;

FIG. 2D-2F are bar graphs showing the improvement in pruritus, erythema, skin and ear secretions exhibited by canines consuming the control pet food composition or a third non-limiting pet food composition according to aspects of the invention;

FIG. 3 is a bar graph of the cytokines levels of canines before and after consuming the control pet food composition or the second exemplary pet food composition in accordance with aspects of the invention;

FIG. 4 is a bar graph of the cytokines levels of canines before and after consuming the control pet food composition or the third exemplary pet food composition according to aspects of the invention;

FIG. 5 is a bar graph showing the abundance level of amino acids exhibited by canines after consuming the control pet food composition or the second exemplary pet food composition in accordance with aspects of the invention; and

FIG. 6 is a bar graph of the amount of the control composition or the second exemplary pet food composition consumed by the canines according to aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both an ancient grain and an amaranth. If a particular pet food care composition recites both an ancient grain and an amaranth, a compound that may be characterized as both an ancient grain and an amaranth will serve only as either an ancient or an amaranth—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions and, particularly, pet food compositions comprising a botanical component. The botanical component comprises a plurality of botanical ingredients. Botanical ingredients are generally ingredients that are derived and/or extracted from plants, such as from roots, stem, fruits, leaves, and/or flowers of plants. The botanical ingredients may be in the form of an aqueous extract, an organic extract, or an oil based extract. A botanical ingredient, such as a botanical extract, may contain a plurality of active compounds, which may be associated with one or more benefits. An extract may optionally contain other non-bioactive compounds, which may or may not be associated with any in-use benefits.

The botanical component may be present in the pet food composition in an amount from about 0.1 to about 15 wt. %, based on the total weight of the pet food composition on a dry matter basis. For example, the pet food composition may contain a botanical component in an amount from about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 15 wt. %, about 0.4 to about 10 wt. %, about 0.4 to about 8 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 15 wt. %, about 0.8 to about 10 wt. %, about 0.8 to about 8 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.3 to about 15 wt. %, about 1.3 to about 10 wt. %, about 1.3 to about 8 wt. %, about 1.3 to about 6 wt. %, about 1.3 to about 5 wt. %, about 1.3 to about 4 wt. %, about 1.3 to about 3 wt. %; from about 1.8 to about 15 wt. %, about 1.8 to about 10 wt. %, about 1.8 to about 8 wt. %, about 1.8 to about 6 wt. %, about 1.8 to about 5 wt. %, about 1.8 to about 4 wt. %, about 1.8 to about 3 wt. %; from about 2.4 to about 15 wt. %, about 2.4 to about 10 wt. %, about 2.4 to about 8 wt. %, about 2.4 to about 6 wt. %, about 2.4 to about 5 wt. %; from about 2.8 to about 15 wt. %, about 2.8 to about 10 wt. %, about 2.8 to about 8 wt. %, about 2.8 to about 6 wt. %, about 2.8 to about 5 wt. %, or any range or subrange thereof, based on the total weight of the pet food composition on a dry matter basis. The pet food compositions may be formulated to contain an effective amount of a botanical component to provide at least a 10% increase in arginine metabolic pathway compounds and/or products thereof, such as ornithine, citrulline, argininosuccinate, and/or hydroxyproline, at about 24 hours after a pet consuming the pet food composition for at least 5 consecutive days. In some preferred embodiments, the pet food composition provides an increase of at least 15%, at least 20%, at least 30%, at least 40%, at least 60%, or at least 80% in arginine metabolic pathway compounds and/or products thereof at about 24 hours after a pet consuming the pet food composition for at least 5, at least 7, at least 10, or at least 42 consecutive days.

The pet food compositions disclosed herein typically comprise a plurality of botanical ingredients selected from cinnamon, green tea, pomegranate, ginger, lime peel, tulsi, and a combination of two or more thereof. Preferably, the pet food composition comprises three to six botanical ingredients, which may be selected from cinnamon, green tea, pomegranate, ginger, lime peel, tulsi, and a combination of two or more thereof. The inventors surprisingly discovered pet food compositions having a botanical component comprising certain combinations of botanical ingredients and/or compounds unexpectedly achieved reductions in symptoms associated with atopic dermatitis in canines consuming such pet food compositions.

In accordance with one aspect of the invention, provided is a pet food composition that includes an effective amount of a plurality of botanical ingredients selected from cinnamon, green tea, pomegranate, ginger, lime peel, tulsi, and a combination of two or more thereof. In some embodiments, the plurality of botanical ingredients includes three, four, five, or six botanical ingredients. For example, the plurality of botanical ingredients may include three to six, three to five, or three or four botanical ingredients. According to one embodiment, the plurality of botanical ingredients may include four to six, four or five, or five or six botanical ingredients.

The botanical component preferably includes one or more botanical ingredients selected from cinnamon, green tea, pomegranate, ginger, lime peel, and tulsi. In some cases, the botanical ingredients may be selected from cinnamon, green tea, pomegranate, ginger, lime peel, and a combination of two or more thereof. In further cases, the botanical ingredients may be selected from cinnamon, green tea, pomegranate, ginger, tulsi, and a combination of two or more thereof. Preferably, the pet food composition comprises two or three botanical ingredients selected from cinnamon, green tea, pomegranate, and ginger. In at least one embodiment, the pet food composition comprises each of the following botanical ingredients: cinnamon, green tea, pomegranate, and ginger.

The pet food compositions may be formulated to include not more than six botanical ingredients. For instance, the pet food composition may have a botanical component consisting of three to six botanical ingredients selected from cinnamon, green tea, pomegranate, ginger, lime peel, tulsi, and a combination of two or more thereof. In some embodiments, the botanical component may consist of three, four, five, six, or any range formed therefrom of botanical ingredients selected from cinnamon, green tea, pomegranate, ginger, lime peel, and/or tulsi. In at least one instance, the botanical component includes botanical ingredients (such as, three or four botanical ingredients) selected from the group consisting of cinnamon, green tea, pomegranate, ginger, and a combination of two or more thereof.

The plurality of botanical ingredients in the pet food composition may include cinnamon and/or cinnamon extract in an amount ranging from about 0.05 to about 2 wt. %, based on the total weight of pet food composition on a dry matter basis. For instance, the amount of cinnamon and/or cinnamon extract present in the pet food composition may be from about 0.05 to about 1.75 wt. %, about 0.05 to about 1.5 wt. %, about 0.05 to about 1.25 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %; from about 0.1 to about 2 wt. %, about 0.1 to about 1.75 wt. %, about 0.1 to about 1.5 wt. %, about 0.1 to about 1.25 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.15 to about 2 wt. %, about 0.15 to about 1.75 wt. %, about 0.15 to about 1.5 wt. %, about 0.15 to about 1.25 wt. %, about 0.15 to about 1 wt. %, about 0.15 to about 0.8 wt. %, about 0.15 to about 0.6 wt. %, about 0.15 to about 0.4 wt. %, about 0.15 to about 0.2 wt. %; from about 0.2 to about 2 wt. %, about 0.2 to about 1.75 wt. %, about 0.2 to about 1.5 wt. %, about 0.2 to about 1.25 wt. %, about 0.2 to about 1 wt. %, about 0.2 to about 0.8 wt. %, about 0.2 to about 0.6 wt. %, about 0.2 to about 0.4 wt. %; from about 0.3 to about 2 wt. %, about 0.3 to about 1.75 wt. %, about 0.3 to about 1.5 wt. %, about 0.3 to about 1.25 wt. %, about 0.3 to about 1 wt. %, about 0.3 to about 0.8 wt. %, about 0.3 to about 0.6 wt. %; from about 0.4 to about 2 wt. %, about 0.4 to about 1.75 wt. %, about 0.4 to about 1.5 wt. %, about 0.4 to about 1.25 wt. %, about 0.4 to about 1 wt. %, about 0.4 to about 0.8 wt. %, about 0.4 to about 0.6 wt. %, or any range or subrange thereof, based on the total weight of the pet food composition on a dry matter basis.

The plurality of botanical ingredients in the pet food composition may include green tea and/or green tea extract in an amount ranging from about 0.05 to about 2 wt. %, based on the total weight of pet food composition on a dry matter basis. For instance, the amount of green tea and/or green tea extract present in the pet food composition may be from about 0.05 to about 1.75 wt. %, about 0.05 to about 1.5 wt. %, about 0.05 to about 1.25 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %; from about 0.1 to about 2 wt. %, about 0.1 to about 1.75 wt. %, about 0.1 to about 1.5 wt. %, about 0.1 to about 1.25 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.15 to about 2 wt. %, about 0.15 to about 1.75 wt. %, about 0.15 to about 1.5 wt. %, about 0.15 to about 1.25 wt. %, about 0.15 to about 1 wt. %, about 0.15 to about 0.8 wt. %, about 0.15 to about 0.6 wt. %, about 0.15 to about 0.4 wt. %, about 0.15 to about 0.2 wt. %; from about 0.2 to about 2 wt. %, about 0.2 to about 1.75 wt. %, about 0.2 to about 1.5 wt. %, about 0.2 to about 1.25 wt. %, about 0.2 to about 1 wt. %, about 0.2 to about 0.8 wt. %, about 0.2 to about 0.6 wt. %, about 0.2 to about 0.4 wt. %; from about 0.3 to about 2 wt. %, about 0.3 to about 1.75 wt. %, about 0.3 to about 1.5 wt. %, about 0.3 to about 1.25 wt. %, about 0.3 to about 1 wt. %, about 0.3 to about 0.8 wt. %, about 0.3 to about 0.6 wt. %; from about 0.4 to about 2 wt. %, about 0.4 to about 1.75 wt. %, about 0.4 to about 1.5 wt. %, about 0.4 to about 1.25 wt. %, about 0.4 to about 1 wt. %, about 0.4 to about 0.8 wt. %, about 0.4 to about 0.6 wt. %, or any range or subrange thereof, based on the total weight of the pet food composition on a dry matter basis.

The plurality of botanical ingredients in the pet food composition may include pomegranate and/or pomegranate extract in an amount ranging from about 0.05 to about 2 wt. %, based on the total weight of pet food composition on a dry matter basis. For instance, the amount of pomegranate and/or pomegranate extract present in the pet food composition may be from about 0.05 to about 1.75 wt. %, about 0.05 to about 1.5 wt. %, about 0.05 to about 1.25 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %; from about 0.1 to about 2 wt. %, about 0.1 to about 1.75 wt. %, about 0.1 to about 1.5 wt. %, about 0.1 to about 1.25 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.15 to about 2 wt. %, about 0.15 to about 1.75 wt. %, about 0.15 to about 1.5 wt. %, about 0.15 to about 1.25 wt. %, about 0.15 to about 1 wt. %, about 0.15 to about 0.8 wt. %, about 0.15 to about 0.6 wt. %, about 0.15 to about 0.4 wt. %, about 0.15 to about 0.2 wt. %; from about 0.2 to about 2 wt. %, about 0.2 to about 1.75 wt. %, about 0.2 to about 1.5 wt. %, about 0.2 to about 1.25 wt. %, about 0.2 to about 1 wt. %, about 0.2 to about 0.8 wt. %, about 0.2 to about 0.6 wt. %, about 0.2 to about 0.4 wt. %; from about 0.3 to about 2 wt. %, about 0.3 to about 1.75 wt. %, about 0.3 to about 1.5 wt. %, about 0.3 to about 1.25 wt. %, about 0.3 to about 1 wt. %, about 0.3 to about 0.8 wt. %, about 0.3 to about 0.6 wt. %; from about 0.4 to about 2 wt. %, about 0.4 to about 1.75 wt. %, about 0.4 to about 1.5 wt. %, about 0.4 to about 1.25 wt. %, about 0.4 to about 1 wt. %, about 0.4 to about 0.8 wt. %, about 0.4 to about 0.6 wt. %, or any range or subrange thereof, based on the total weight of the pet food composition on a dry matter basis.

The plurality of botanical ingredients in the pet food composition may include ginger and/or ginger extract in an amount ranging from about 0.005 to about 1 wt. %, based on the total weight of pet food composition on a dry matter basis. For instance, the amount of ginger and/or ginger extract present in the pet food composition may be from about 0.005 to about 0.8 wt. %, about 0.005 to about 0.6 wt. %, about 0.005 to about 0.4 wt. %, about 0.005 to about 0.2 wt. %, about 0.005 to about 0.1 wt. %; from about 0.01 to about 1.25 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.015 to about 1 wt. %, about 0.015 to about 0.8 wt. %, about 0.015 to about 0.6 wt. %, about 0.015 to about 0.4 wt. %, about 0.015 to about 0.2 wt. %, about 0.015 to about 0.1 wt. %; from about 0.02 to about 1 wt. %, about 0.02 to about 0.8 wt. %, about 0.02 to about 0.6 wt. %, about 0.02 to about 0.4 wt. % about 0.02 to about 0.2 wt. %, about 0.02 to about 0.1 wt. %; from about 0.03 to about 1 wt. %, about 0.03 to about 0.8 wt. %, about 0.03 to about 0.6 wt. %, about 0.03 to about 0.4 wt. %, about 0.03 to about 0.2 wt. %, about 0.03 to about 0.1 wt. %; from about 0.04 to about 1 wt. %, about 0.04 to about 0.8 wt. %, about 0.04 to about 0.6 wt. %, about 0.04 to about 0.4 wt. %, about 0.04 to about 0.2 wt. %, about 0.04 to about 0.1 wt. %, or any range or subrange thereof, based on the total weight of the pet food composition on a dry matter basis.

The plurality of botanical ingredients in the pet food composition may include lime peel and/or lime peel extract in an amount ranging from about 0.2 to about 3 wt. %, based on the total weight of pet food composition on a dry matter basis. For instance, the amount of lime peel and/or lime peel extract present in the pet food composition may be about 0.2 to about 3 wt. %, about 0.2 to about 2 wt. %, about 0.2 to about 1.5 wt. %, about 0.2 to about 1 wt. %; from about 0.5 to about 3 wt. %, about 0.5 to about 2 wt. %, about 0.5 to about 1.5 wt. %, about 0.5 to about 1 wt. %; from about 0.75 to about 3 wt. %, about 0.75 to about 2 wt. %, about 0.75 to about 1.5 wt. %; from about 1 to about 3 wt. %, about 1 to about 2 wt. %, or about 1 to about 1.5 wt. %, including any range or subrange thereof, based on the total weight of the pet food composition on a dry matter basis.

The plurality of botanical ingredients in the pet food composition may include tulsi and/or tulsi extract in an amount ranging from about 0.0006 to about 1 wt. %, based on the total weight of pet food composition on a dry matter basis. For instance, the amount of tulsi and/or tulsi extract present in the pet food composition may be from about 0.0006 to about 0.6 wt. %, about 0.0006 to about 0.2 wt. %, about 0.0006 to about 0.1 wt. %, about 0.0006 to about 0.08 wt. %, about 0.0006 to about 0.06 wt. %, about 0.0006 to about 0.04 wt. %, about 0.0006 to about 0.02 wt. %; from about 0.001 to about 1 wt. %, about 0.001 to about 0.6 wt. %, about 0.001 to about 0.2 wt. %, about 0.001 to about 0.1 wt. %, about 0.001 to about 0.08 wt. %, about 0.001 to about 0.06 wt. %, about 0.001 to about 0.04 wt. %, about 0.001 to about 0.02 wt. %; from about 0.005 to about 1 wt. %, about 0.005 to about 0.6 wt. %, about 0.005 to about 0.2 wt. %, about 0.005 to about 0.1 wt. %, about 0.005 to about 0.08 wt. %, about 0.005 to about 0.06 wt. %, about 0.005 to about 0.04 wt. %, about 0.005 to about 0.02 wt. %; from about 0.01 to about 1 wt. %, about 0.01 to about 0.6 wt. %, about 0.01 to about 0.2 wt. %, about 0.01 to about 0.1 wt. %, about 0.01 to about 0.08 wt. %, about 0.01 to about 0.06 wt. %, about 0.01 to about 0.04 wt. %; from about 0.05 to about 1 wt. %, about 0.05 to about 0.6 wt. %, about 0.05 to about 0.2 wt. %, about 0.05 to about 0.1 wt. %; from about 0.1 to about 1 wt. %, about 0.1 to about 0.6 wt. %, or about 0.1 to about 0.2 wt. %, including ranges and subranges thereof, based on the total weight of the pet food composition on a dry matter basis.

In at least one preferable embodiment, the pet food composition comprises a plurality of botanical ingredients and/or extracts chosen from about 0.2 wt. % of cinnamon, about 0.1 wt. % of green tea (about 0.2 wt. % of a green tea having a 50% concentration of green tea), about 0.3 wt. % of pomegranate, about 0.03 of ginger (dry), about 1 wt. % of lime peel (dry), and about 0.003 of Tulsi, and a combination of two or more thereof. In yet another embodiment, the pet food composition comprises about 0.2 wt. % of cinnamon, about 0.1 wt. % of green tea (about 0.2 wt. % of a green tea having a 50% concentration of green tea), about 0.3 wt. % of pomegranate, and about 0.03 of ginger (dry).

The botanical component may include the plurality of botanical ingredients in any of the amounts discussed above and, optionally, in certain weight ratios. For example, the pet food composition may include a weight ratio of cinnamon to green tea of from about 1:5 to about 7:1, about 1:3 to about 5:1, about 1:2 to about 3:1, or about 1:1 to about 2:1. Additionally or alternatively, the pet food composition may include a weight ratio of pomegranate to cinnamon of from about 1:5 to about 7:1, about 1:3 to about 5:1, about 1:2 to about 3:1, or about 1:1 to about 2:1. In some embodiments, the pet food composition includes a weight ratio of green tea to ginger of about 20:1 to about 1:1, about 16:1 to about 3:1, about 12:1 to about 5:1, or about 9:1 to about 5:1. The pet food composition may optionally be formulated to have a weight ratio of cinulin to green tea to pomegranate to ginger of from 3-15:3-15:5-20:1, or 6-10:6-10:8-12:1.

Additionally or alternatively, the pet food compositions may include coumarins (1,2-benzopyrone or 2H-1-benzopyran-2-one), type-A polymers, polyphenols (such as, epigallocatechin gallate and catechins), ellagic acid, gingerols, derivatives thereof, or combinations of two or more thereof. The pet food compositions may include various beneficial combinations of coumarins, type-A polymers, epigallocatechin gallate, catechins, polyphenols, ellagic acid, gingerols, including all of the foregoing compounds. For example, may include a combination of coumarins and type-A polymers. Type-A polymers are typically type-A polymers of flavonoids, such as double-linked procyanidin type-A polymers of flavonoids. The pet food compositions, in some cases, includes polyphenols not limited to, but including epigallocatechin gallate, catechin, glycoside structures of kaempferol, and quercetin. In at least one preferred embodiment, the pet food composition comprises one or more polyphenols chosen from epigallocatechin gallate and catechin. In some instances, the pet food composition includes epigallocatechin gallate, catechins, polyphenols. In at least one embodiment, the pet food composition includes ellagic acid, gingerols, and/or derivatives thereof. Yet additional embodiments, coumarins, type-A polymers, and ellagic acid can be incorporated into the pet food composition. In further embodiments, the pet food composition includes coumarins, type-A polymers, and gingerols. In at least one additional embodiment, the pet food composition may include epigallocatechin gallate, catechins, polyphenols, and ellagic acid.

The pet food compositions may include coumarins, type-A polymers, epigallocatechin gallate, catechins, polyphenols, ellagic acid, gingerols, and/or derivatives thereof in various amounts. For example, the amount of coumarins and/or derivatives thereof present in the pet food composition may be from about 0.0001 to about 0.02 wt. %, about 0.0001 to about 0.01 wt. %, about 0.0001 to about 0.007 wt. %, about 0.0001 to about 0.004 wt. %, about 0.0001 to about 0.002 wt. %; from about 0.0005 to about 0.02 wt. %, about 0.0005 to about 0.01 wt. %, about 0.0005 to about 0.007 wt. %, about 0.0005 to about 0.004 wt. %, about 0.0005 to about 0.002 wt. %; from about 0.001 to about 0.02 wt. %, about 0.001 to about 0.01 wt. %, about 0.001 to about 0.007 wt. %, about 0.001 to about 0.004 wt. %, about 0.001 to about 0.002 wt. %; from about 0.0012 to about 0.02 wt. %, about 0.0012 to about 0.01 wt. %, about 0.0012 to about 0.007 wt. %, about 0.0012 to about 0.004 wt. %, about 0.0012 to about 0.002 wt. %; from about 0.0014 to about 0.02 wt. %, about 0.0014 to about 0.01 wt. %, about 0.0014 to about 0.007 wt. %, about 0.0014 to about 0.004 wt. %, about 0.0014 to about 0.002 wt. %; from about 0.002 to about 0.02 wt. %, about 0.002 to about 0.01 wt. %, about 0.002 to about 0.007 wt. %, about 0.002 to about 0.004 wt. %, or any ranges and subranges thereof, based on the total weight of the pet food composition on a dry matter basis.

The pet food compositions may include type-A polymers and/or derivatives thereof in an amount from about 0.006 to about 0.1 wt. %, based on the total weight of the pet food composition on a dry matter basis. In some instances, the amount of type-A polymers and/or derivatives thereof present in the pet food composition is from about 0.006 to about 0.08 wt. %, about 0.006 to about 0.05 wt. %, about 0.006 to about 0.01 wt. %, about 0.006 to about 0.008 wt. %; from about 0.01 to about 0.1 wt. %, about 0.01 to about 0.05 wt. %; from about 0.03 to about 0.1 wt. %, about 0.03 to about 0.05 wt. %; from about 0.06 to about 0.1 wt. %; from about 0.08 to about 0.1 wt. %, or any range or subrange thereof, based on the total weight of the pet food composition on a dry matter basis.

The pet food compositions may include caffeine in an amount up to a maximum of 0.004 wt. %, based on the total weight of the pet food composition on a dry matter basis. Preferably, the pet food compositions are substantially free or free of caffeine. For example, the amount of caffeine in the pet food composition may be about 0.0005 to 0.004 wt. %, about 0.0005 to about 0.002 wt. %, about 0.0005 to about 0.001 wt. %, about 0.0005 to about 0.0008 wt. %; from about 0.0008 to 0.004 wt. %, about 0.0008 to about 0.002 wt. %, about 0.0008 to about 0.001 wt. %; from about 0.001 to 0.004 wt. %, about 0.001 to about 0.002 wt. %; from about 0.002 to 0.004 wt. %, or any range or subrange thereof, based on the total weight of the pet food composition on a dry matter basis.

The pet food compositions may include epigallocatechin gallate in an amount from about 0.06 to about 1 wt. %, based on the total weight of the pet food composition on a dry matter basis. In some instances, the amount of epigallocatechin gallate present in the pet food composition is from about 0.1 to about 1 wt. %, about 0.2 to about 1 wt. %, about 0.3 to about 1 wt. %, about 0.4 to about 1 wt. %, about 0.5 to about 1 wt. %, about 0.6 to about 1 wt. %, about 0.7 to about 1 wt. %, about 0.8 to about 1 wt. %; from about 0.06 to about 0.8 wt. %, about 0.1 to about 0.8 wt. %, about 0.2 to about 0.8 wt. %, about 0.3 to about 0.8 wt. %, about 0.4 to about 0.8 wt. %, about 0.5 to about 0.8 wt. %, about 0.6 to about 0.8 wt. %; from about 0.06 to about 0.7 wt. %, about 0.1 to about 0.7 wt. %, about 0.2 to about 0.7 wt. %, about 0.3 to about 0.7 wt. %, about 0.4 to about 0.7 wt. %, about 0.5 to about 0.7 wt. %; from about 0.06 to about 0.6 wt. %, about 0.1 to about 0.6 wt. %, about 0.2 to about 0.6 wt. %, about 0.3 to about 0.6 wt. %; from about 0.06 to about 0.5 wt. %, about 0.1 to about 0.5 wt. %, about 0.2 to about 0.5 wt. %, about 0.3 to about 0.5 wt. %, or any range and subrange thereof, based on the total weight of the pet food composition on a dry matter basis.

The pet food compositions may include catechins and/or derivatives thereof in an amount from about 0.1 to about 0.75 wt. %, based on the total weight of the pet food composition on a dry matter basis. For example, the amount of catechins present in the pet food compositions may be from about 0.1 to about 0.6 wt. %, about 0.1 to about 0.5 wt. %, about 0.1 to about 0.4 wt. %, about 0.1 to about 0.3 wt. %, about 0.1 to about 0.2 wt. %; from about 0.15 to about 0.75 wt. %, about 0.15 to about 0.6 wt. %, about 0.15 to about 0.5 wt. %, about 0.15 to about 0.4 wt. %, about 0.15 to about 0.3 wt. %; from about 0.2 to about 0.75 wt. %, about 0.2 to about 0.6 wt. %, about 0.2 to about 0.5 wt. %, about 0.2 to about 0.4 wt. %, about 0.2 to about 0.3 wt. %, or any range or subrange thereof, based on the total weight of the pet food composition on a dry matter basis.

The pet food compositions may include polyphenols and/or derivatives thereof in an amount from about 0.1 to about 1 wt. %, based on the total weight of the pet food composition on a dry matter basis. In some cases, the amount of polyphenols and/or derivatives thereof present in the pet food composition is from about 0.1 to about 1 wt. %, about 0.1 to about 0.8 wt. %, about 0.1 to about 0.6 wt. %, about 0.1 to about 0.5 wt. %, about 0.1 to about 0.4 wt. %, about 0.1 to about 0.3 wt. %, about 0.1 to about 0.2 wt. %; from about 0.15 to about 1 wt. %, about 0.15 to about 0.8 wt. %, about 0.15 to about 0.6 wt. %, about 0.15 to about 0.5 wt. %, about 0.15 to about 0.4 wt. %, about 0.15 to about 0.3 wt. %; from about 0.2 to about 1 wt. %, about 0.2 to about 0.8 wt. %, about 0.2 to about 0.6 wt. %, about 0.2 to about 0.5 wt. %, about 0.2 to about 0.4 wt. %, about 0.2 to about 0.3 wt. %; from about 0.4 to about 1 wt. %, about 0.4 to about 0.8 wt. %, about 0.4 to about 0.6 wt. %, including any range or subrange thereof, based on the total weight of the pet food composition on a dry matter basis.

The pet food compositions may include ellagic acid in an amount from about 0.005 to about 0.5 wt. %, based on the total weight of the pet food composition on a dry matter basis. In some cases, the amount of ellagic acid present in the pet food composition is from about 0.005 to about 0.4 wt. %, about 0.005 to about 0.3 wt. %, about 0.005 to about 0.2 wt. %, about 0.005 to about 0.1 wt. %, about 0.005 to about 0.05 wt. %; from about 0.01 to about 0.5 wt. %, about 0.01 to about 0.4 wt. %, about 0.01 to about 0.3 wt. %, about 0.01 to about 0.2 wt. %, about 0.01 to about 0.1 wt. %, about 0.01 to about 0.05 wt. %; from about 0.02 to about 0.5 wt. %, about 0.02 to about 0.4 wt. %, about 0.02 to about 0.3 wt. %, about 0.02 to about 0.2 wt. %, about 0.02 to about 0.1 wt. %, about 0.02 to about 0.05 wt. %; from about 0.04 to about 0.5 wt. %, about 0.04 to about 0.4 wt. %, about 0.04 to about 0.3 wt. %, about 0.04 to about 0.2 wt. %, about 0.04 to about 0.1 wt. %; from about 0.06 to about 0.5 wt. %, about 0.06 to about 0.4 wt. %, about 0.06 to about 0.3 wt. %, about 0.06 to about 0.2 wt. %; from about 0.08 to about 0.5 wt. %, about 0.08 to about 0.4 wt. %, about 0.08 to about 0.3 wt. %, about 0.08 to about 0.2 wt. %; from about 0.15 to about 0.5 wt. %, about 0.15 to about 0.4 wt. %, about 0.15 to about 0.3 wt. %, or any range or subrange thereof, based on the total weight of the pet food composition on a dry matter basis.

The pet food compositions may include gingerols and/or derivatives thereof in an amount of about 0.0001 to about 0.003 wt. %, based on the total weight of the pet food compositions on a dry matter basis. For example, the amount of gingerols and/or derivatives thereof present in the pet food compositions may be from about 0.0001 to about 0.002 wt. %, about 0.0001 to about 0.001 wt. %, about 0.0001 to about 0.0008 wt. %, about 0.0001 to about 0.0005 wt. %, about 0.0001 to about 0.0003 wt. %; from about 0.0002 to about 0.003 wt. %, about 0.0002 to about 0.002 wt. %, about 0.0002 to about 0.001 wt. %, about 0.0002 to about 0.0008 wt. %, about 0.0002 to about 0.0005 wt. %, about 0.0002 to about 0.0003 wt. %; from about 0.0003 to about 0.003 wt. %, about 0.0003 to about 0.002 wt. %, about 0.0003 to about 0.001 wt. %, about 0.0003 to about 0.0008 wt. %, about 0.0003 to about 0.0005 wt. %; from about 0.0006 to about 0.003 wt. %, about 0.0006 to about 0.002 wt. %, about 0.0006 to about 0.001 wt. %; from about 0.001 to about 0.003 wt. %, about 0.001 to about 0.002 wt. %, or any range or subrange thereof, based on the total weight of the pet food composition on a dry matter basis.

The botanical component may include polyphenols, such as one or more of coumarins, type-A polymers, epigallocatechin gallate, catechins, ellagic acid, gingerols, and/or derivatives thereof in any of the amounts discussed above and, optionally, in certain weight ratios. For example, the pet food compositions may, optionally, be formulated to have a weight ratio of epigallocatechin gallate to catechins of from about 1:10 to about 10:1. In some instances, the weight ratio of epigallocatechin gallate to catechins may be from about 1:7 to about 7:1, about 1:4 to about 4:1, about 1:3 to about 3:1, or about 1:2 to about 2:1. The pet food composition may have a weight ratio of catechins to ellagic acid of from about 1:5 to about 15:1, about 1:2 to about 15:1, about 1:1 to about 10:1, or about 1:1 to about 5:1. Additionally or alternatively, the pet food composition may have a weight ratio of ellagic acid to gingerols of about 100:1 to about 3:1, about 75:1 to about 5:1, about 50:1 to about 5:1, about 75:1 to about 15:1, or about 50:1 to about 15:1. In some cases, the pet food composition has a weight ratio of coumarins to gingerols of about 10:1 to 1:10, about 10:1 to about 1:1, about 7:1 to about 1:1, or about 6:1 to about 2:1.

According to another aspect of the invention, provided is a pet food composition that including a fat; a protein; and a botanical component. The botanical component typically comprises three to six botanical ingredients selected from about 0.05 to about 2 wt. % of cinnamon; from about 0.05 to about 2 wt. % of green tea; from about 0.05 to about 2 wt. % of pomegranate; from about 0.005 to about 1 wt. % of ginger; from about 0.2 to about 3 wt. % of lime peel extract; and from about 0.0006 to about 1 wt. % of tulsi.

Suitable components, such as those listed below, may be included or excluded from the formulations for the pet food compositions depending on the specific combination of other ingredients and the form of the pet food compositions. In some embodiments, the pet food compositions disclosed herein may be in the form of a standalone pet food, as a supplement to pet food, as a pet food treat, or the like.

The pet food compositions are formulated to include fat in an amount that may vary, but typically is in the range of about 8 to about 50 wt. %, endpoints included, based on the total weight of the pet food composition on a dry matter basis. For example, the pet food composition may include fat in an amount ranging from about 10 to about 50 wt. %, about 12 to about 50 wt. %, about 14 to about 50 wt. %, about 16 to about 50 wt. %, about 18 to about 50 wt. %, about 20 to about 50 wt. %, about 22 to about 50 wt. %, about 24 to about 50 wt. %; from about 8 to about 40 wt. %, about 10 to about 40 wt. %, about 12 to about 40 wt. %, about 14 to about 40 wt. %, about 16 to about 40 wt. %, about 18 to about 40 wt. %, about 20 to about 40 wt. %, about 22 to about 40 wt. %, about 24 to about 40 wt. %; from about 8 to about 35 wt. %, about 10 to about 35 wt. %, about 12 to about 35 wt. %, about 14 to about 35 wt. %, about 16 to about 35 wt. %, about 18 to about 35 wt. %, about 20 to about 35 wt. %, about 22 to about 35 wt. %, about 24 to about 35 wt. %; about 8 to about 30 wt. %, about 10 to about 30 wt. %, about 12 to about 30 wt. %, about 14 to about 30 wt. %, about 16 to about 30 wt. %, about 18 to about 30 wt. %, about 20 to about 30 wt. %, about 22 to about 30 wt. %, about 24 to about 30 wt. %; from about 8 to about 27 wt. %, about 10 to about 27 wt. %, about 12 to about 27 wt. %, about 14 to about 27 wt. %, about 16 to about 27 wt. %, about 18 to about 27 wt. %, about 20 to about 27 wt. %, about 22 to about 27 wt. %; from about 8 to about 24 wt. %, about 10 to about 24 wt. %, about 12 to about 24 wt. %, about 14 to about 24 wt. %, about 16 to about 24 wt. %, about 18 to about 24 wt. %, about 20 to about 24 wt. %; from about 8 to about 22 wt. %, about 10 to about 22 wt. %, about 12 to about 22 wt. %, about 14 to about 22 wt. %, about 16 to about 22 wt. %, about 18 to about 22 wt. %; from about 8 to about 22 wt. %, about 10 to about 22 wt. %, about 12 to about 22 wt. %, about 14 to about 22 wt. %, about 16 to about 22 wt. %, about 18 to about 22 wt. %; from about 8 to about 20 wt. %, about 10 to about 20 wt. %, about 12 to about 20 wt. %, about 14 to about 20 wt. %, about 16 to about 20 wt. %, including ranges and subranges thereof, based on the total weight of the pet food composition on a dry matter basis.

The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. The fat may be incorporated completely within the food composition, deposited on the outside of the pet food composition, or a mixture of the two methods. In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

The fat may comprise dietary fats, such as triglycerides. In some embodiments, the triglyceride may comprise about 20 to about 100%, about 40 to about 100%, about 50 to about 100%, about 60 to about 100%, about 70 to about 100%, about 80 to about 100%, about 90 to about 100%, of the total amount of fat in the pet food composition. In further embodiments, the triglyceride comprises about 20 to about 95%, about 40 to about 95%, about 50 to about 95%, about 60 to about 95%, about 70 to about 95%, about 80 to about 95%, about 90 to about 95%, of the total amount of fat in the pet food composition. In additional embodiments, the triglyceride comprises about 20 to about 90%, about 40 to about 90%, about 50 to about 90%, about 60 to about 90%, about 70 to about 90%, about 80 to about 90%, of the total amount of fat in the pet food composition. In yet further embodiment, the triglyceride comprises about 20 to about 80%, about 40 to about 80%, about 50 to about 80%, about 60 to about 80%, about 70 to about 80%, of the total amount of fat in the pet food composition.

The triglyceride may include one or more constituents that comprise a fatty acid(s) component/moiety. For example, the triglyceride may include one, two, or three aplanatic chains that are selected from fatty acid component. Non-limiting examples of fatty acid components include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetraenoic acid, and a combination of two or more thereof. The fatty acid(s) moieties may be a polyunsaturated fatty acid, such as an omega-3 fatty acid, an omega-6 fatty acid, or a combination of two or more thereof. Non-limiting examples of omega-3 fatty acid components include those selected from linolenic acid, stearidonic acid, eicosatetraenoic acid, eicosapentaenoic acid, docosapentaenoic acid, docosahexaenoic acid, and a combination of two or more thereof. Examples of omega-6 fatty acid moieties include linolenic acid, calendic acid, eicosadienoic acid, arachidonic acid, docosadienoic acid, adrenic acid, osbond acid, tetracosatetraenoic acid, tetracosapentaenoic acid, or a combination of two or more thereof.

The triglyceride may be selected from triglycerides having at least one aliphatic carbon chain comprised of 6 to 10 carbons. For example, the triglyceride may have one, two, or three aliphatic carbons chains of 6 to 10 carbons. In some embodiments, the triglyceride has an aliphatic carbon chain of 6 carbons, 7 carbons, 8 carbons, 9 carbons, and/or 10 carbons. For example, the aliphatic carbon chain of the medium chain triglyceride may include from 6 to 9 carbons, 6 to 8 carbons, 6 or 7 carbons; 7 to 10 carbons, 7 to 9 carbons, 7 or 8 carbons; 8 to 10 carbons, or 8 or 9 carbons, or 9 or 10 carbons. In some embodiments, the triglyceride(s) comprises an aliphatic carbon chain having 6 carbons, 8 carbons, or 10 carbons. For example, the triglyceride may have an aliphatic carbon chain selected from caprylate, caprate, and/or decanoate.

The pet food compositions may optionally contain one or more fatty acid(s). Non-limiting examples of fatty acids include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetraenoic acid, and a combination of two or more thereof. The fatty acid(s) may be a polyunsaturated fatty acid, such as an omega-3 fatty acid, an omega-6 fatty acid, or a combination of two or more thereof. Non-limiting examples of omega-3 fatty acids include those selected from linolenic acid, stearidonic acid, eicosatetraenoic acid, eicosapentaenoic acid, docosapentaenoic acid, docosahexaenoic acid, and a combination of two or more thereof. The pet food composition may include linolenic acid, eicosapentaenoic acid, docosahexaenoic acid, or a combination of two or more thereof. In at least one embodiment, the pet food composition comprises alpha-linolenic acid and/or gamma-linolenic acid. Additionally or alternatively, the polyunsaturated fatty acid may comprise an omega-6 fatty acids. Examples of omega-6 fatty acid include linolenic acid, calendic acid, eicosadienoic acid, arachidonic acid, docosadienoic acid, adrenic acid, osbond acid, tetracosatetraenoic acid, tetracosapentaenoic acid, or a combination of two or more thereof. In some embodiments, the polyunsaturated fatty acid comprises an omega-6 fatty acid selected from linolenic acid, arachidonic acid, and a combination of two or more thereof.

The pet food composition may be formulated to have a weight ratio of omega-3 fatty acids to omega-6 fatty acids of from about 0.5:1 to about 7:1. In some embodiments, the pet food composition has a weight ratio of omega-3 fatty acids to omega-6 fatty acids of from about 0.5:1 to about 6:1, about 0.5:1 to about 5:1, about 0.5:1 to about 4:1, about 0.5:1 to about 3:1, about 0.5:1 to about 2.5:1, about 0.5:1 to about 2:1, about 0.5:1 to about 1.5:1, or about 0.5:1 to about 1:1; from about 1:1 to about 6:1, about 1:1 to about 5:1, about 1:1 to about 4:1, about 1:1 to about 3:1, about 1:1 to about 2.5:1, about 1:1 to about 2:1, about 1:1 to about 1.5:1, or about 1:1 to about 1:1, including ranges or subranges thereof.

The fatty acid(s) present in the pet food composition may be in an amount from about 0.1 to about 5 wt. %, based on the total weight of the pet food composition on a dry matter basis. For example, the pet food composition may contain fatty acid(s) in an amount from about 0.1 to about 4 wt. %, about 0.1 to about 3 wt. %, about 0.1 to about 2 wt. %, about 0.1 to about 1 wt. %; from about 0.2 to about 5 wt. %, about 0.2 to about 4 wt. %, about 0.2 to about 3 wt. %, about 0.2 to about 2 wt. %, about 0.2 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 0.75 to about 5 wt. %, about 0.75 to about 4 wt. %, about 0.75 to about 3 wt. %, about 0.75 to about 2 wt. %, about 0.75 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subranges thereof, based on the total weight of the pet food composition on a dry matter basis.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the compositions is crude fat. Crude fat may be included into the compositions in the amounts disclosed above with respect of the total fat, such as from about 8 to about 50 wt. %, based on the total weight of the pet food composition on a dry matter basis. In some embodiments, the pet food composition comprises crude fat in an amount of about 10 to about 40 wt. %, about 12 to about 35 wt. %, about 14 to about 30 wt. %, about 16 to about 24 wt. %, based on the total weight of the pet food composition on a dry matter basis. In some cases, it may be preferable that about 50 wt. % or more, about 60 wt. % or more, about 70 wt. % or more, about 80 wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food compositions typically include protein in an amount ranging from about 15 to about 55 wt. %, based on the total weight of the pet food composition on a dry matter basis. In some instances, the total amount of protein in the pet food composition is in a range from about 15 to about 50 wt. %, about 15 to about 48 wt. %, about 15 to about 46 wt. %, about 15 to about 44 wt. %, about 15 to about 42 wt. %, about 15 to about 40 wt. %, about 15 to about 38 wt. %, about 15 to about 36 wt. %, about 15 to about 34 wt. %; from about 20 to about 55 wt. %, about 20 to about 50 wt. %, about 20 to about 48 wt. %, about 20 to about 46 wt. %, about 20 to about 44 wt. %, about 20 to about 42 wt. %, about 20 to about 40 wt. %, about 20 to about 38 wt. %, about 20 to about 36 wt. %, about 20 to about 34 wt. %; from about 25 to about 55 wt. %, about 25 to about 50 wt. %, about 25 to about 48 wt. %, about 25 to about 46 wt. %, about 25 to about 44 wt. %, about 25 to about 42 wt. %, about 25 to about 40 wt. %, about 25 to about 38 wt. %, about 25 to about 36 wt. %, about 25 to about 34 wt. %; from about 30 to about 55 wt. %, about 30 to about 50 wt. %, about 30 to about 48 wt. %, about 30 to about 46 wt. %, about 30 to about 44 wt. %, about 30 to about 42 wt. %, about 30 to about 40 wt. %, about 30 to about 38 wt. %, or about 30 to about 36 wt. %, including ranges and subranges therebetween, based on the total weight of the pet food composition on a dry matter basis.

The protein of the pet food composition comprises one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine, and a combination of two or more thereof. The pet food composition may comprise two or more amino acids. For instance, the pet food composition may include two or more, three or more, four or more, five or more, six or more, seven or more, eight or more amino acids. In some embodiments, the pet food composition includes glycine and proline, and optionally one or more additional amino acids.

In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

A portion of the protein in the composition may be digestible protein. For example, the composition may include an amount of protein, where about 40 wt. % or more, about 50 wt. % or more, about 60 wt. % or more, about 70 wt. % or more, about 80 wt. % or more, about 90 wt. % or more, about 95 wt. % or more, about 98 wt. % or more, or about 99 wt. % or more of the protein is digestible protein. In some embodiments, e.g., when the composition desirable promotes weight loss, the portion of protein that is digestible protein is about 60 wt. % or less, about 50 wt. % or less, about 40 wt. % or less, about 30 wt. % or less, about 20 wt. % or less, or about 10 wt. % or less, based on the total amount of protein in the pet food composition on a dry matter basis. In further embodiment, the amount of protein that is digestible protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, about 70 to about 90 wt. %, including ranges and subranges therein, based on the total amount of protein in the pet food composition on a dry matter basis.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food compositions are typically formulated to include fiber in an amount from about 10 to about 60 wt. %, based on the total weight of the pet food composition on a dry matter basis. For instance, the amount of fiber present in the pet food composition may be from about 10 to about 55 wt. %, about 10 to about 50 wt. %, about 10 to about 45 wt. %, about 10 to about 40 wt. %, about 10 to about 35 wt. %, about 10 to about 30 wt. %; from about 15 to about 60 wt. %, about 15 to about 55 wt. %, about 15 to about 50 wt. %, about 15 to about 45 wt. %, about 15 to about 40 wt. %, about 15 to about 35 wt. %, about 15 to about 30 wt. %; from about 20 to about 60 wt. %, about 20 to about 55 wt. %, about 20 to about 50 wt. %, about 20 to about 45 wt. %, about 20 to about 40 wt. %, about 20 to about 35 wt. %, about 20 to about 30 wt. %; from about 25 to about 60 wt. %, about 25 to about 55 wt. %, about 25 to about 50 wt. %, about 25 to about 45 wt. %, about 25 to about 40 wt. %, about 25 to about 35 wt. %; from about 30 to about 60 wt. %, about 30 to about 55 wt. %, about 30 to about 50 wt. %, about 30 to about 45 wt. %, about 30 to about 40 wt. %; from about 35 to about 60 wt. %, about 35 to about 55 wt. %, about 35 to about 50 wt. %, about 35 to about 45 wt. %; from about 40 to about 60 wt. %, about 40 to about 55 wt. %, about 40 to about 50 wt. %; from about 45 to about 60 wt. %, about 45 to about 55 wt. %; from about 50 to about 60 wt. %, including ranges and subranges thereof, based on the total weight of the pet food composition on a dry matter basis.

The total amount of fiber present in the pet food composition generally comprises an amount of crude fiber and dietary fiber. The amount of crude fiber and/or dietary fiber may be present in the pet food compositions in any of the above amounts disclosed for the total amount of fiber. Crude fiber includes indigestible components contained in cell walls and cell contents of plants such as grains, e.g., hulls of grains such as rice, corn, and beans.

Dietary fiber refers to components of a plant that are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole-wheat products, wheat oat, corn bran, flax seed, grapes, celery, green beans, cauliflower, potato skins, fruit skins (e.g., pear skin), vegetable skins, peanut hulls, almond shell, walnut shell, pecan shell, citrus pulp, beet pulp, and soy fiber. In some embodiments, the dietary fiber may be chosen from pecan shell, citrus pulp, beet pulp, pear skin, and a combination of two or more thereof. Crude fiber includes indigestible components contained in cell walls and cell contents of plants such as grains, for example, hulls of grains such as rice, corn, and beans.

In some embodiments, the pet food composition has a weight ratio of insoluble fiber to soluble fiber from about 20:1 to about 8:1. For example, the pet food composition may have a weight ratio of insoluble fiber to soluble fiber from about 18:1 to about 8:1, about 16:1 to about 8:1, about 14:1 to about 8:1, about 12:1 to about 8:1, about 10:1 to about 8:1. The pet food composition may have a weight ratio of insoluble fiber to soluble fiber of about 11:1.

Additionally and/or alternatively, the fiber component of the pet food composition may comprise an acid detergent fiber and/or a neutral detergent fiber. In some instances, the pet food composition includes an acid detergent fiber and a neutral detergent fiber, e.g., in an amount that is from about 1 to about 20 wt. %, based on the total weight of the pet food composition on a dry matter basis. For example, the pet food composition may include one of an acid detergent fiber and/or a neutral detergent fiber in an amount from about 1 to about 15 wt. %, about 1 to about 12 wt. %, about 1 to about 10 wt. %, about 1 to about 8 wt. %, about 1 to about 6 wt. %, about 1 to about 5 wt. %, about 1 to about 3 wt. %; from about 3 to about 15 wt. %, about 3 to about 12 wt. %, about 3 to about 10 wt. %, about 3 to about 8 wt. %, about 3 to about 6 wt. %, about 3 to about 5 wt. %; about 5 to about 15 wt. %, about 5 to about 12 wt. %, about 5 to about 10 wt. %, about 5 to about 8 wt. %, about 5 to about 6 wt. %; about 7 to about 15 wt. %, about 7 to about 12 wt. %, about 7 to about 10 wt. %, about 7 to about 8 wt. %; about 9 to about 15 wt. %, about 9 to about 12 wt. %, about 9 to about 10 wt. %; about 11 to about 15 wt. %, about 11 to about 13 wt. %, or about 12 to about 15 wt. %, including ranges and subranges thereof, based on the total weight of the pet food composition on a dry matter basis. In some embodiments, the pet food composition has a weight ratio of the acid detergent fiber to the neutral detergent fiber of from about 3:1 to about 1:3. In some embodiments, the weight ratio of the acid detergent fiber to the neutral detergent fiber is from about 2:1 to about 1:2 or about 1:1.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

The pet food composition may include carbohydrates, e.g., in an amount up to about 65 wt. %, based on the total weight of the pet food composition on a dry matter basis. The term “carbohydrate” as used herein includes polysaccharides (e.g., starches and dextrins) and sugars (e.g., sucrose, lactose, maltose, glucose, and fructose) that are metabolized for energy when hydrolyzed. One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

Generally, carbohydrate percentage can be calculated as nitrogen free extract (“NFE”), which can be calculated as follows: NFE %=100%−(moisture %)−(protein %)−(fat %)−(ash %)−(crude fiber %) The amount of carbohydrate, e.g., calculated as NFE, present in the composition may be from an amount up to about 65 wt. %, an amount up to about 60 wt. %, an amount up to about 55 wt. %, an amount up to about 50 wt. %, an amount up to about 45 wt. %, an amount up to about 40 wt. %, an amount up to about 35 wt. %, an amount up to about 30 wt. %, an amount up to about 25 wt. %, an amount up to about 20 wt. %, an amount up to about 15 wt. %, an amount up to about 10 wt. %, an amount up to about 5 wt. %; about 1 to about 65 wt. %, about 1 to about 55 wt. %, about 1 to about 50 wt. %, about 1 to about 45 wt. %, about 1 to about 40 wt. %, about 1 to about 35 wt. %; about 1 to about 30 wt. %, about 1 to about 25 wt. %, about 1 to about 20 wt. %, about 1 to about 15 wt. %, about 1 to about 10 wt. %, about 1 to about 5 wt. %; about 5 to about 65 wt. %, about 5 to about 55 wt. %, about 5 to about 50 wt. %, about 5 to about 45 wt. %, about 5 to about 40 wt. %, about 5 to about 35 wt. %; about 5 to about 30 wt. %, about 5 to about 25 wt. %, about 5 to about 20 wt. %, about 5 to about 15 wt. %; about 10 to about 65 wt. %, about 10 to about 55 wt. %, about 10 to about 50 wt. %, about 10 to about 45 wt. %, about 10 to about 40 wt. %, about 10 to about 35 wt. %; about 10 to about 30 wt. %, about 10 to about 25 wt. %; about 15 to about 65 wt. %, about 15 to about 55 wt. %, about 15 to about 50 wt. %, about 15 to about 45 wt. %, about 15 to about 40 wt. %, about 15 to about 35 wt. %; about 15 to about 30 wt. %; about 20 to about 65 wt. %, about 20 to about 55 wt. %, about 20 to about 50 wt. %, about 20 to about 45 wt. %, about 20 to about 40 wt. %, about 20 to about 35 wt. %; about 25 to about 65 wt. %, about 25 to about 55 wt. %, about 25 to about 50 wt. %, about 25 to about 45 wt. %, about 25 to about 40 wt. %, about 25 to about 35 wt. %; about 30 to about 65 wt. %, about 30 to about 55 wt. %, about 30 to about 50 wt. %, about 30 to about 45 wt. %; about 35 to about 65 wt. %, about 35 to about 55 wt. %, about 35 to about 50 wt. %; about 40 to about 65 wt. %, about 40 to about 55 wt. %, about 45 to about 65 wt. %, about 45 to about 55 wt. %; or about 50 to about 65 wt. %, including ranges and subranges thereof, based on the total weight of the pet composition on a dry matter basis.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof. The amount of arginine present in the composition may be about 0.01 to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about 0.5 wt. %, about 1.0 wt. %, about 1.4 weight %, about 1.44 wt. %, about 1.8 wt. %, or about 2.0 wt. %, based on the total weight of the composition on a dry matter basis. The arginine present in the composition may be L-arginine, D-arginine, or a mixture thereof.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include glucosamine, chondroitin sulfate, vegetable extracts, herbal extracts, etc.

The concentration of such additives in the pet food composition typically can be up to about 5 wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about 0.01 to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; or about 4 to about 5 wt. %, based on the total weight of the pet food composition on a dry matter basis. In some embodiments, the concentration of such additives (particularly where such additives are primarily nutritional balancing agents, such as vitamins and minerals) is from about 0 to about 2.0% by weight, based on the total weight of the pet food composition on a dry matter basis. The amount of additives comprising vitamins may be in addition to the amount of vitamin B discussed above. In some embodiments, the concentration of such additives (again, particularly where such additives are primarily nutritional balancing agents) is from about 0 to about 1.0% by weight, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat foods, for example, are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitines, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with a further aspect of the invention, a method is provided for pruritus, erythema, alopecia, or skin and ear secretion in atopic dermatitis canine, the method comprising administering a pet food composition disclosed herein. The method may include providing and/or feeding the canine a pet food composition disclosed herein. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

### EXAMPLES

### Example 1—Pet Food Compositions

Three pet food compositions (Example Compositions A-C) were prepared by combining three botanical components with a base pet food composition (Control Composition 1). Control Composition 1 also served as a positive control for the following examples. The three botanical components, which were used for preparing Example Composition A-C, are shown in Table 1, below. The nutritional value of Example Compositions A-C and Control Composition 1 in Table 2, below.

TABLE 1

| | | Ex. | Ex. | Ex. | Control |
|---|---|---|---|---|---|
| | | Comp. A | Comp. B | Comp. C | Comp. 1 |
| | | (wt. %) | (wt. %) | (wt. %) | (wt. %) |
| Base | Rice (brewers) | 34 | 33 | 34 | 34 |
| Component | Chicken Meal | 14 | 14 | 14 | 14 |
| | Sorghum (whole) | 12 | 12 | 12 | 12 |
| | Barley | 8.6 | 8.6 | 8.6 | 8.6 |
| | Pea Protein | 4.1 | 4.1 | 4.1 | 4.1 |
| | Egg Dried | 4 | 4 | 4 | 4 |
| | Soybean Oil | 3.2 | 2 | 3 | 3 |
| | Flax Seed | 3.1 | 3.1 | 3.1 | 3.1 |
| | Palatant | 4.5 | 4.5 | 4.5 | 4.5 |
| | Oat (groats) | 2.7 | 2.7 | 2.7 | 2.7 |
| | Beet Pulp | 2.5 | 2.5 | 2.5 | 2.5 |
| | Fish oil | 1.9 | 1.9 | 1.9 | 1.9 |
| | Lactic acid | 1 | 1 | 1 | 1 |
| | Chicken Fat acidified | 1.1 | 1 | 1 | 1 |
| | Potassium chloride | 0.6 | 0.4 | 0.4 | 0.4 |
| | Fruit and Vegetable Blend | 0.5 | 0.5 | 0.5 | 0.5 |
| | Lipoic acid, alpha, 5% | 0.3 | 0.3 | 0.3 | 0.3 |
| | Choline Chloride | 0.2 | 0.2 | 0.2 | 0.2 |
| | Vitamin E (oil) (29 wt. % | 0.26 | 0.18 | 0.18 | 0.18 |
| | concentration of Vitamin E) | | | | |
| | Sodium Chloride | 0.21 | 0.21 | 0.21 | 0.21 |
| | Vitamin (premix) | 0.16 | 0.09 | 0.09 | 0.09 |
| | Calcium Carbonate | 0.1595 | 0.1595 | 0.1595 | 0.1595 |
| | Mineral (premix) | 0.078 | 0.078 | 0.078 | 0.078 |
| | Taurine | 0.07 | 0.07 | 0.07 | 0.07 |
| | Antioxidant, Verdilox | 0.04 | 0.04 | 0.04 | 0.04 |
| | Natural Flavor | 0.03 | 0.03 | 0.03 | 0.03 |
| Botanical | Cinnamon | 0.1-0.6 | 0.1-0.6 | 0.1-0.8 | — |
| Component | Green Tea | 0.1-0.6 | 0.1-0.6 | 0.1-0.8 | — |
| | (50 wt. % concentration of Green | | | | |
| | Tea) | | | | |
| | Pomegranate Extract | 0.2-0.8 | 0.2-0.8 | — | — |
| | Ginger | 0.02-0.1 | 0.02-0.1 | — | — |
| | Extract (dry) | | | | |
| | Lime peel dried | 0.5-2 | — | — | — |
| | Tulsi Extract | 0.001-0.01 | — | — | — |

TABLE 2

| | Ex. | Ex. | Ex. | Control |
|---|---|---|---|---|
| | Comp. A | Comp. B | Comp. C | Comp. 1 |
| | (wt. %) | (wt. %) | (wt. %) | (wt. %) |
| Ash | 5.1 | 5.1 | 5.1 | 5.3 |
| Crude Fat | 12.6 | 12.6 | 12.6 | 12.9 |
| Crude Fiber | 1.4 | 1.4 | 1.4 | 1.4 |
| Total Dietary Fiber | 6.1 | 6.1 | 6.1 | 6.1 |
| Moisture | 8 | 8 | 8 | 8 |
| Crude Protein | 21 | 21 | 21.1 | 21 |
| Carbohydrates | 52 | 51.9 | 51.8 | 51.3 |
| Omega-3 Sum | 1.6 | 1.6 | 1.6 | 1.7 |
| Omega-6 Sum | 3 | 3.5 | 3.4 | 3.7 |

### Example 2—Evaluation of Ex. Comps. A and B

Example Compositions A and B were evaluated in comparison to positive control, Control Composition 1. Specifically, 26 clinically diagnosed dermatological canines of both genders having ages ranging from 5.9 years to 16.9 years and body weights ranging from 8.3 to 21.5 kg were evaluated. All of the canines were being neutered or spayed.

The canines were sorted into three groups, with Group 1 having 7 dogs, Group 2 having 9 dogs, and Group 3 having 10 dogs. The dogs of Group 1 were fed Control Composition 1 for 60 days, the dogs of Group 2 were fed Composition 2 for 60 days, and the dogs of Group 3 were fed Example Composition A for 60 days, as part of a feed regimen. None of the canines underwent a pre-feed phase before receiving one of the pet food compositions for the 60 day period. Clinical assessment of dermatological health was assessed by a veterinarian using a prescribed questionnaire before starting the 60 day feed regimen to establish a baseline and at the end of 60 day feed regimen to determine the severity of pruritus, erythema, skin and ear secretions and alopecia.

### Example 3—Evaluation of Ex. Comp. B

Example Composition B was evaluated in comparison to Control Composition 1 to assess the effect of Example Composition B on canines suffering from dermatological problems. Specifically, 19 canines of mixed gender with ages ranging from 9.0 years to 16.9 years and body weights ranging from 7.5 to 25.6 kg were evaluated in this Example. All canines were canines clinically diagnosed with dermatological problems and were neutered or spayed.

The canines were separated into two groups by matching age and gender. The first group of canines was fed Example Composition B for a period of 42 days while the second group was fed Control Composition 1 for a period of 42 days as a first feed regimen. After the 42 days of the first feed regimen, the first group was fed Control Composition 1 for a period of 42 days and the second group was fed Example Composition B for a period of 42 days, as a second feed regimen. The canines did not undergo a pre-feed or washout feed regimen.

Blood samples were collected from each canine before starting the first feed regimen, after the first feed regimen, and after the second regimen. Clinical assessment of dermatological health was assessed by a veterinarian using a prescribed questionnaire before the first feed regimen, after the first feed regimen, and after the second regimen, to determine the severity of pruritus, erythema, skin and ear secretions and alopecia.

Additionally, the palatability of Example Composition B and Control Composition 1 was assessed to determine if the four botanical ingredients effected the palatability of Example Composition B. Although a two-bowl PAL test was not performed for this Example, the amount of Example Composition B and Control Composition 1 consumed by the canines was monitored to assess palatability and rejection of food.

### Example 4—Evaluation of Ex. Comp. C

Example Composition C was evaluated in comparison to Control Composition 1 to assess the effect of Example Composition C on canines suffering from dermatological problems.

Specifically, 20 canines having ages ranging from 9.0 years to 16.4 years and body weights ranging from 7.5 to 25.6 kg were evaluated in this Example. All canines were mixed gender, neutered or spayed, and clinically diagnosed with dermatological problems.

The 20 canines were then separated into two groups by matching age and gender. The first group was fed Example Composition C for a period of 42 days while the second group was fed Control Composition 1 for a period of 42 days as a first feed regimen. After the 42 days of the first feed regimen, the first group was fed Control Composition 1 for a period of 42 days and the second group was fed Example Composition C for a period of 42 days, as a second feed regimen. The canines did not undergo a pre-feed or washout feed regimen before the first or second feed regimens.

Blood samples were collected from each canine before starting the first feed regimen, after the first feed regimen, and after the second regimen for cytokines analyses. Clinical assessment of dermatological health was assessed by a veterinarian using a prescribed questionnaire before starting the first feed regimen, after the first feed regimen, and after the second regimen to determine the severity of pruritus, erythema, skin and ear secretions and alopecia.

### Example 5—Comparison of Pet Food Compositions Evaluated in Examples 2-4

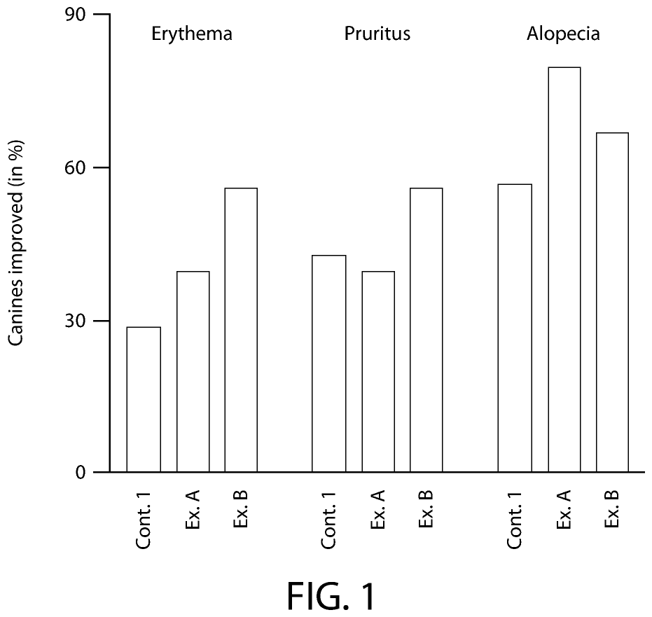

As discussed above, Example 2 evaluated Example Compositions A and B in comparison to Control Composition 1, which was a positive control pet food composition. Based on Example 2, Example Composition B increased the response rate (improvement %) of pruritus and erythema compared with Example Composition A and Control Composition 1 (see FIG. 1). However, Example Composition A increased the response rate (improvement %) of alopecia more than by Example Composition B and significantly more than Control Composition 1. No differences were observed for skin and ear secretions between all pet food compositions. Among the dermatological problems, specifically, pruritus and erythema are key characteristics of atopic dermatitis in dogs and are a primary reason for veterinary visits.

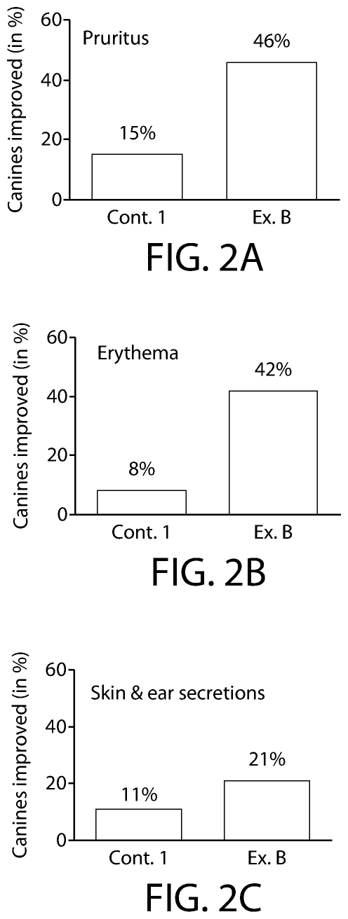

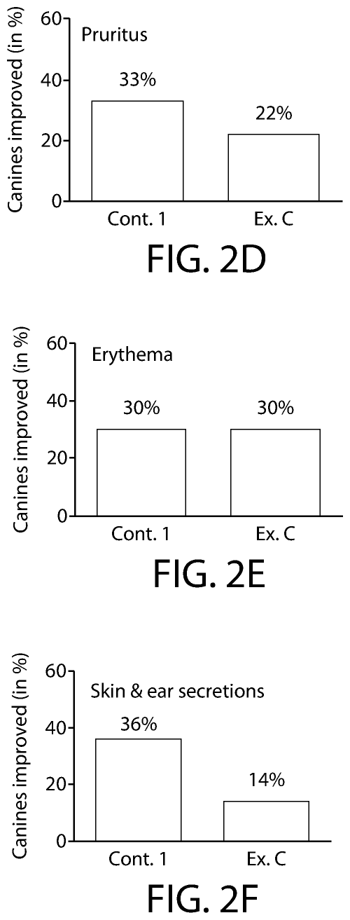

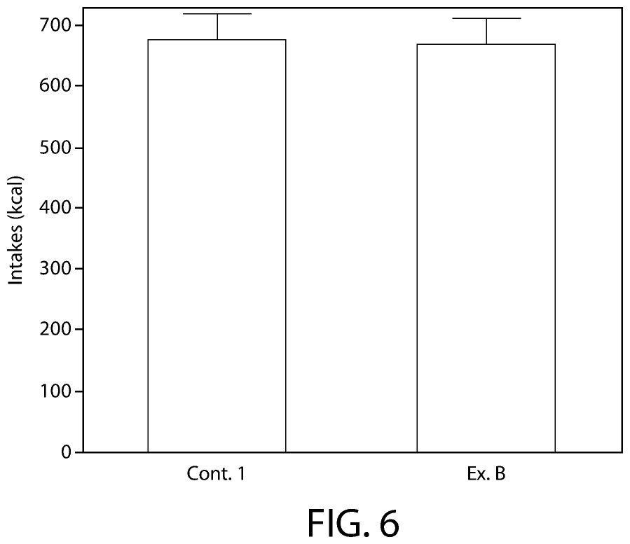

In Example 3, Example Composition B increased the response rate (improvement %) of pruritus, erythema and skin and ear secretions of dogs by 46%, 42% and 21% respectively, when compared with Control Composition 1 (see FIGS. 2A-2C). Additionally, although it conventionally known that botanical ingredients can negatively affect palatability, there was no significant difference in the consumption of Example Composition B as compared to Control Composition 1 (see FIG. 6), and none of the canines rejected Example Composition B.

Regarding Example 4, Example Composition C was evaluated in composition to Control Composition 1. It was determined that Example Composition C did not show any improvement of pruritus, erythema and skin and ear secretions of dogs compared with Control Composition 1 (see FIG. 2D-2F). It was not particularly surprising that Control Composition 1 exhibited better performance in alleviating dermatological symptoms than Example Composition C as Control Composition 1 was a positive control.

Overall, Example Composition B reduced pruritus and erythema robustly when compared with Control Composition 1 and Example Compositions A and C, which would significantly improve the quality of a pet's life by reducing scratching and other behavior problems associated with chronic pruritus, which was not expected by the inventors.

### Example 6—Assessment of Systemic Immunity of Pet Food Compositions

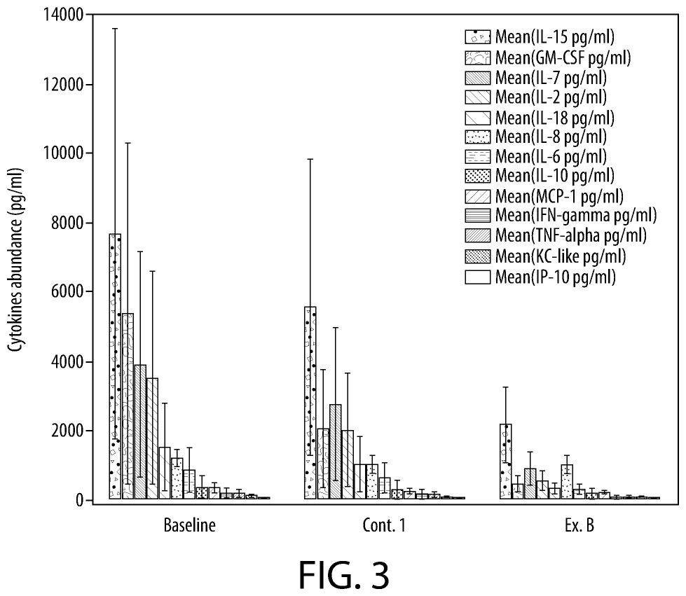

The blood samples from the canines in Examples 3 and 4 were evaluated to assess the change in systemic immunity based on consumption of the pet food compositions. Specifically, the blood samples were evaluated to determine the change in circulating levels of 13 cytokines. In Example 3, the canines consuming Example Composition B exhibited a decreasing trend of all cytokines levels compared with the baseline and the canines consuming Control Composition 1 (see FIG. 3). However, we did not observe a statistical significance for all cytokines between Example Composition B, Example Composition C, and Control Composition 1, which is likely due to higher inter individual variation. Specifically, it has been demonstrated that the cytokines IL-15, IL-7, IL-2, IL-18, IL-8 and IL-6 are highly expressed and associated with the severity of atopic dermatitis patients compared with healthy individuals. Example Composition B provided a considerable decrease in trend for these cytokine levels in canines consuming Example Composition B when compared with Control Composition 1.

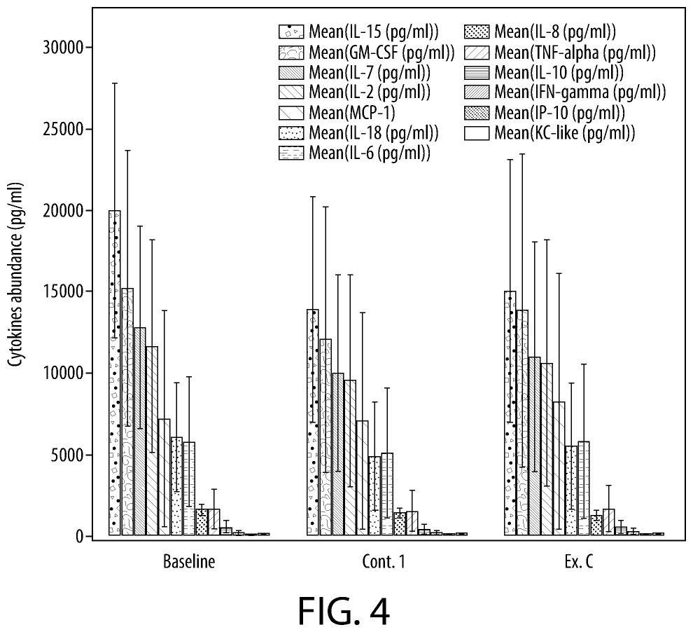

In Example 4, it was determined that none of the cytokine levels showed a decreasing trend in canines consuming Example Composition C when compared to Control Composition 1. Control Composition 1 exhibited a decreasing trend of some cytokines levels in canines consuming Control Composition 1 when compared with the baseline (see FIG. 4).

Overall, the circulating cytokines expression analysis suggests that Example Composition B reduces cytokine levels that are associated with the severity of atopic dermatitis to improve the quality of pet's life by reducing scratching.

In addition, a non-targeted metabolites analysis in serum samples collected from the Example 3 was preformed and it was determined that Example Composition B provide a significant decrease of circulating acylcarnitines levels (see Table 3, below) compared with the baseline but not with Control Composition 1. Acylcarnitines are the products of incomplete betaoxidation of fatty acids, which are conventionally believed to have the potential to activate proinflammatory signaling pathways.

TABLE 3

| | Overall model | | | |
|---|---|---|---|---|
| | (treatment effect) | | | |
| | FDR corrected | Mean ± SE | | |
| Biochemical | P value | Baseline | Cont. 1 | Ex. B |
| 3-hydroxybutyrylcarnitine (1) | 0.0244 | 0.152 ± 0.2^{A} | 0.366 ± 0.19^{B} | −0.255 ± 0.18^{B} |
| Acetylcarnitine (C2) | 0.00107 | 0.161 ± 0.07^{A} | 0.112 ± 0.06^{B} | −0.107 ± 0.06^{B} |
| Arachidonoylcarnitine (C20:4) | 9.96E−09 | 0.494 ± 0.08^{A} | −0.188 ± 0.09^{B} | −0.472 ± 0.12^{B} |
| Arachidoylcarnitine (C20)* | 5.77E−09 | 0.49 ± 0.1^{A} | −0.167 ± 0.09^{B} | −0.43 ± 0.1^{B} |
| Behenoylcarnitine (C22)* | 0.000567 | −0.117 ± 0.07^{A} | −0.401 ± 0^{B} | −0.393 ± 0^{B} |
| Cerotoylcarnitine (C26)* | 0.00427 | −0.013 ± 0.1^{A} | −0.231 ± 0.08^{B} | −0.311 ± 0.08^{B} |
| Cis-4-decenoylcarnitine | 0.00219 | 0.177 ± 0.15^{A} | −0.265 ± 0.11^{B} | −0.351 ± 0.12^{B} |
| (C10:1) | | | | |
| decanoylcarnitine (C10) | 0.000909 | 0.262 ± 0.13^{A} | −0.359 ± 0.17^{B} | −0.611 ± 0.18^{B} |
| Dihomo-linolenoylcarnitine | 3.14E−09 | 0.349 ± 0.07^{A} | −0.232 ± 0.08^{B} | −0.341 ± 0.09^{B} |
| (20:3n3 or 6)* | | | | |
| Dihomo-linoleoylcarnitine | 1.38E−10 | 0.38 ± 0.05^{A} | −0.188 ± 0.07^{B} | −0.375 ± 0.09^{B} |
| (C20:2)* | | | | |
| Eicosenoylcarnitine (C20:1)* | 7.93E−09 | 0.369 ± 0.04^{A} | −0.121 ± 0.07^{B} | −0.289 ± 0.07^{B} |
| Hexanoylcarnitine (C6) | 0.0273 | 0.187 ± 0.1^{A} | −0.037 ± 0.08^{B} | −0.07 ± 0.09^{B} |
| Laurylcarnitine (C12) | 0.00127 | 0.268 ± 0.11^{A} | −0.136 ± 0.12^{B} | −0.452 ± 0.13^{B} |
| Lignoceroylcarnitine (C24)* | 1.50E−07 | 0.329 ± 0.06^{A} | −0.102 ± 0.05^{B} | −0.245 ± 0.06^{B} |
| Linoleoylcarnitine (C18:2)* | 7.17E−06 | 0.15 ± 0.05^{A} | −0.13 ± 0.05^{B} | −0.275 ± 0.06^{B} |
| Margaroylcarnitine* | 0.00028 | 0.242 ± 0.08^{A} | −0.097 ± 0.07^{B} | −0.308 ± 0.09^{B} |
| Myristoleoylcarnitine (C14:1)* | 0.0499 | 0.118 ± 0.11^{A} | −0.076 ± 0.1^{AB} | −0.295 ± 0.1^{B} |
| Myristoylcarnitine (C14) | 0.000451 | 0.193 ± 0.07^{A} | −0.034 ± 0.06^{B} | −0.205 ± 0.08^{B} |
| Nervonoylcarnitine (C24:1)* | 0.0331 | −0.151 ± 0.08^{A} | −0.43 ± 0.07^{B} | −0.398 ± 0.06^{B} |
| Octanoylcarnitine (C8) | 0.00253 | 0.268 ± 0.11^{A} | −0.1 ± 0.09^{B} | −0.156 ± 0.09^{B} |
| Oleoylcarnitine (C18) | 6.37E−07 | 0.264 ± 0.05^{A} | −0.097 ± 0.06^{B} | −0.232 ± 0.07 |
| Palmitoylcarnitine (C16) | 6.40E−07 | 0.203 ± 0.04^{A} | −0.124 ± 0.05^{B} | −0.226 ± 0.06^{B} |
| Stearoylcarnitine (C18) | 2.63E−09 | 0.49 ± 0.07^{A} | −0.161 ± 0.07^{B} | −0.303 ± 0.07^{B} |

Example Composition B also showed a significant increase of circulating long chain polyunsaturated fatty acids (PUFAs), such as eicosapentaenoic acid (EPA) and docosahexaenoic acid (DHA), compared with the baseline, not with Control Composition 1. Long chain PUFAs are generally antioxidants, anti-inflammatory, and can attenuate food allergy (see Table 4, below).

TABLE 4

| | Overall model | | | |
|---|---|---|---|---|
| | (treatment effect) | | | |
| | FDR corrected | Mean ± SE | | |
| Biochemical | P value | Baseline | Cont. 1 | Ex. B |
| Docosahexaenoate (DHA; | 5.87E−12 | −1.038 ± 0.14^{B} | 0.203 ± 0.05^{A} | 0.135 ± 0.08^{A} |
| 22:6n3) | | | | |
| Docosapentaenoate (DPA; | 0.000169 | −0.481 ± 0.11^{B} | 0.183 ± 0.08^{A} | 0.073 ± 0.12^{A} |
| 22:5n3) | | | | |
| Eicosapentaenoate (EPA; | 3.08E−11 | −1.903 ± 0.11^{B} | 0.165 ± 0.08^{A} | 0.141 ± 0.1^{A} |
| 20:5n3) | | | | |
| Heneicosapentaenoate | 9.46E−10 | −1.88 ± 0.16^{B} | 0.221 ± 0.1^{A} | 0.186 ± 0.14^{A} |
| (21:5n3) | | | | |
| Linolenate (18:3n3 or 3n6) | 0.000401 | −0.494 ± 0.11^{B} | 0.075 ± 0.08^{A} | 0.031 ± 0.11^{A} |

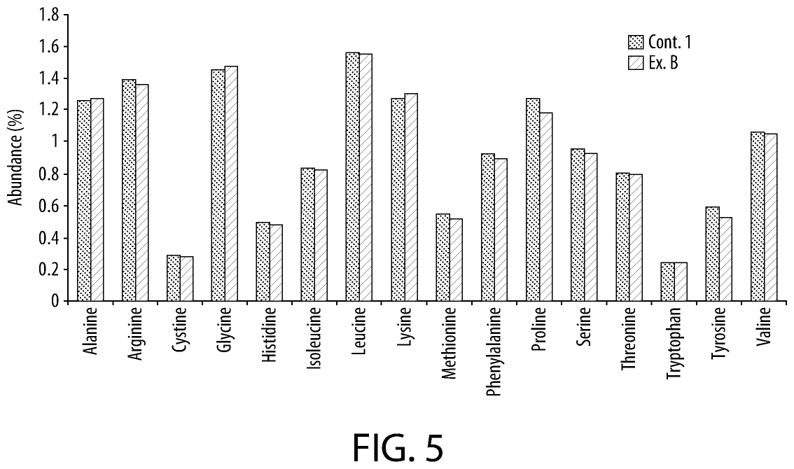

Canines consuming Example Composition B exhibited a significant increase in the compounds utilized by the arginine metabolic pathway as well as its products, such as ornithine, citrulline, argininosuccinate, and hydroxyproline compared with the baseline, but not in comparison to canines consuming Control Composition 1 (see Table 5). This was unexpected because the dietary amino acids profiles in Example Composition B and Control Composition 1 are similar (see FIG. 5).

TABLE 5

| | Overall model | | | |
|---|---|---|---|---|
| | (treatment effect) | | | |
| | FDR corrected | Mean ± SE | | |
| Biochemical | P value | Baseline | Cont. 1 | Ex. B |
| Arginine | 0.048 | −0.09 ± 0.04^{B} | −0.019 ± 0.04^{A} | −0.011 ± 0.03^{A} |
| Argininosuccinate | 0.0293 | −0.267 ± 0.1^{B} | 0.097 ± 0.09^{A} | −0.016 ± 0.13^{A} |
| Citrulline | 0.000759 | −0.062 ± 0.05^{B} | 0.04 ± 0.05^{A} | 0.092 ± 0.05^{A} |
| Homoarginine | 4E−16 | −1.087 ± 0.1^{B} | 0.113 ± 0.05^{A} | 0.192 ± 0.06^{A} |

Example Composition B provided a significant reduction of short chain length (<24) sphingomyelins and ceramides compared with the baseline, although not when compared with Control Composition 1 (see Tables 6 and 7, below). Increasing short chain length sphingomyelins and ceramides play an important role in impaired skin membrane barrier function in atopic dermatitis (AD). Accordingly, the inventors believe that reducing short chain sphingomyelins and ceramides probably plays a role in improving skin lipid layer homeostasis to improve skin membrane barrier function.

TABLE 6

| | Overall model | | | |
|---|---|---|---|---|
| | (treatment effect) | | | |
| | FDR corrected | Mean ± SE | | |
| Biochemical | P value | Baseline | Cont. 1 | Ex. B |
| Sphingomyelin (d18:1/22:2, | 1.67E−10 | −0.281 ± 0.09^{B} | 0.129 ± 0.07^{A} | 0.109 ± 0.07^{A} |
| d18:2/22:1, d16:1/24:2)* | | | | |
| Sphingomyelin (d18:2/14:0, | 0.00046 | −0.167 ± 0.11^{B} | 0.157 ± 0.1^{A} | 0.092 ± 0.09^{A} |
| d18:1/14:1)* | | | | |
| Sphingomyelin (d18:2/21:0, | 0.000478 | −0.173 ± 0.07^{B} | 0.117 ± 0.08^{A} | 0.065 ± 0.07^{A} |
| d16:2/23:0)* | | | | |
| Sphingomyelin (d18:2/23:1)* | 3.6E−10 | −0.409 ± 0.08^{B} | 0.214 ± 0.07^{A} | 0.106 ± 0.07^{A} |
| Sphingomyelin (d18:2/24:2)* | 6.78E−12 | −0.386 ± 0.11^{B} | 0.241 ± 0.09^{A} | 0.238 ± 0.08^{A} |
| Sphingomyelin (d18:1/20:1, | 6.17E−07 | −0.136 ± 0.07^{B} | 0.076 ± 0.06^{A} | 0.078 ± 0.06^{A} |
| d18:2/20:0)* | | | | |
| Palmitoyl dihydrosphingomyelin | 0.0288 | 0.165 ± 0.06^{A} | 0.089 ± 0.07^{AB} | −0.042 ± 0.05^{B} |
| (d18:0/16:0)* | | | | |
| Sphinganine | 0.00327 | 0.007 ± 0.13^{A} | −0.495 ± 0.03^{B} | −0.351 ± 0.08^{B} |
| Sphingosine | 0.00144 | 0.296 ± 0.08^{A} | −0.2 ± 0.07^{B} | −0.126 ± 0.1^{B} |
| Behenoyl dihydrosphingomyelin | 0.0119 | 0.139 ± 0.07^{A} | 0.02 ± 0.1^{A} | −0.263 ± 0.08^{B} |
| (d18:0/22:0)* | | | | |
| Behenoyl sphingomyelin | 0.00177 | 0.095 ± 0.05^{A} | 0.007 ± 0.04^{AB} | −0.093 ± 0.04^{B} |
| (d18:1/22:0)* | | | | |
| Palmitoyl sphingomyelin | 9.83E−10 | 0.201 ± 0.04^{A} | −0.001 ± 0.04^{B} | −0.069 ± 0.04^{B} |
| (d18:1/16:0) | | | | |
| Sphingomyelin (d17:1/16:0, | 0.000224 | 0.074 ± 0.07^{A} | −0.014 ± 0.06^{B} | −0.124 ± 0.06^{C} |
| d18:1/15:0, d16:1/17:0)* | | | | |
| Sphingomyelin (d18:0/18:0, | 0.00355 | 0.41 ± 0.07^{A} | −0.062 ± 0.12^{B} | −0.283 ± 0.07^{B} |
| d19:0/17:0)* | | | | |
| Sphingomyelin (d18:1/18:1, | 8.79E−14 | 0.228 ± 0.05^{A} | −0.124 ± 0.05^{B} | −0.19 ± 0.06^{B} |
| d18:2/18:0) | | | | |
| Sphingomyelin (d18:1/20:2, | 0.0175 | 0.072 ± 0.15^{A} | −0.182 ± 0.14^{B} | −0.373 ± 0.15^{B} |
| d18:2/20:1, d16:1/22:2)* | | | | |
| Sphingomyelin (d18:1/21:0, | 0.0393 | 0.089 ± 0.09^{A} | −0.004 ± 0.07^{AB} | −0.156 ± 0.06^{B} |
| d17:1/22:0, d16:1/23:0)* | | | | |
| Sphingomyelin (d18:2/16:0, | 4.59E−08 | 0.142 ± 0.06^{A} | 0.047 ± 0.06^{B} | −0.096 ± 0.06^{B} |
| d18:1/16:1)* | | | | |
| Sphingomyelin (d18:2/18:1)* | 6.05E−08 | 0.301 ± 0.07^{A} | −0.004 ± 0.05^{B} | −0.109 ± 0.07^{B} |
| Stearoyl sphingomyelin | 1.65E−05 | 0.138 ± 0.04^{A} | −0.016 ± 0.04^{B} | −0.073 ± 0.04^{B} |
| (d18:1/18:0) | | | | |

TABLE 7

| | Overall model | | | |
|---|---|---|---|---|
| | (treatment effect) | | | |
| | FDR corrected | Mean ± SE | | |
| Biochemical | P value | Baseline | Cont. 1 | Ex. B |
| Ceramide (d18:1/17:0, | 4.59E−08 | 0.174 ± 0.11^{A} | −0.544 ± 0.11^{B} | −0.381 ± 0.14^{B} |
| d17:1/18:0)* | | | | |
| Lactosyl-N-palmitoyl- | 0.032485161 | 0.103 ± 0.09^{A} | 0 ± 0.08^{AB} | −0.041 ± 0.08^{B} |
| sphingosine (d18:1/16:0) | | | | |
| N-palmitoyl-sphingosine | 0.001288443 | 0.202 ± 0.06^{A} | −0.061 ± 0.05^{B} | −0.025 ± 0.06^{B} |
| (d18:1/16:0) | | | | |
| N-stearoyl-sphingadienine | 1.31E−14 | 0.321 ± 0.07^{A} | −1.012 ± 0.11^{B} | −0.87 ± 0.14^{B} |
| (d18:2/18:0)* | | | | |
| N-stearoyl-sphingosine | 2.88E−15 | 0.518 ± 0.05^{A} | −0.192 ± 0.06^{B} | −0.19 ± 0.07^{B} |
| (d18:1/18:0)* | | | | |
| Glycosyl-N-stearoyl- | 3.35E−07 | 0.431 ± 0.08^{A} | −0.131 ± 0.06^{B} | −0.159 ± 0.06^{B} |
| sphingosine (d18:1/18:0) | | | | |

1-28. (canceled)

29. A pet food composition comprising:
an effective amount of a plurality of botanical ingredients selected from cinnamon, green tea, pomegranate, ginger, lime peel, tulsi, and a combination of two or more thereof.

30. The pet food composition according to claim 29, wherein the plurality of botanical ingredients comprises three to six botanical ingredients selected from cinnamon, green tea, pomegranate, ginger, lime peel, and tulsi.

31. The pet food composition according to claim 29, wherein the plurality of botanical ingredients comprises from four to six botanical ingredients selected from cinnamon, green tea, pomegranate, ginger, lime peel, and tulsi.

32. The pet food composition according to claim 29, wherein the plurality of botanical ingredients comprises four botanical ingredients selected from cinnamon, green tea, pomegranate, ginger, lime peel, and tulsi.

33. The pet food composition according to claim 29, wherein the plurality of botanical ingredients comprises cinnamon and pomegranate.

34. The pet food composition according to claim 29, wherein the plurality of botanical ingredients comprises green tea and ginger.

35. The pet food composition according to claim 29, wherein the plurality of botanical ingredients comprises pomegranate, green tea, and ginger.

36. The pet food composition according to claim 29, wherein the plurality of botanical ingredients comprises from about 0.05 to about 2 wt. % of cinnamon, based on the total weight of the pet food composition.

37. The pet food composition according claim 29, further comprising coumarins and type-A polymers.

38. The pet food composition according to claim 29, further comprising at least one of:
from about 0.0001 to about 0.02 wt. % of coumarins, based on the total weight of the petfood composition; and
from about 0.006 to about 0.1 wt. % of type-A polymers, based on the total weight of the pet food composition.

39. The pet food composition according to claim 29, wherein the plurality of botanical ingredients comprises from about 0.05 to about 2 wt. % of green tea, based on the total weight of the pet food composition.

40. The pet food according to claim 29, wherein the pet food composition comprises 0.004 wt. % or less of caffeine, based on the total weight of the pet food composition.

41. The pet food composition according to claim 29, further comprising at least one of:
from about 0.06 to about 1 wt. % of epigallocatechin gallate, based on the total weight of the pet food composition;
from about 0.1 to about 0.75 wt. % of catechins, based on the total weight of the pet food composition; and
from about 0.1 to about 1 wt. % of polyphenols, based on the total weight of the pet food composition.

42. The pet food composition according to claim 29, further comprising epigallocatechin gallate, catechins, and polyphenols.

43. The pet food composition according to claim 29, further comprising ellagic acid, optionally in an amount of from about 0.005 to about 0.5 wt. %, based on the total weight of the pet food composition.

44. The pet food composition according to claim 29, comprising about 0.0001 to about 0.003 wt. % of gingerols and/or derivatives thereof, based on the total weight of the pet food composition.

45. A pet food composition comprising:
a fat;
a protein; and
a botanical component, the botanical component comprising three to six botanical ingredients selected from:
from about 0.05 to about 2 wt. % of cinnamon;
from about 0.05 to about 2 wt. % of green tea;
from about 0.05 to about 2 wt. % of pomegranate;
from about 0.005 to about 1 wt. % of ginger;
from about 0.2 to about 3 wt. % of lime peel extract; and
from about 0.0006 to about 1 wt. % of tulsi,
wherein all weight percentages are based on the total weight of the pet food composition.

46. The pet food composition according to claim 45, wherein the botanical component consists essentially of three to six botanical ingredients selected from:
from about 0.05 to about 2 wt. % of cinnamon;
from about 0.05 to about 2 wt. % of green tea;
from about 0.05 to about 2 wt. % of pomegranate;
from about 0.005 to about 1 wt. % of ginger;
from about 0.2 to about 3 wt. % of lime peel extract; and
from about 0.0006 to about 1 wt. % of tulsi.

47. The pet food composition according to claim 45, wherein the botanical component consists essentially of:
from about 0.05 to about 2 wt. % of cinnamon;
from about 0.05 to about 2 wt. % of green tea;
from about 0.05 to about 2 wt. % of pomegranate; and
from about 0.005 to about 1 wt. % of ginger.

48. The pet food composition according to claim 45, wherein the pet food composition comprises three or more compounds selected from the group consisting of coumarins, type-A polymers, epigallocatechin gallate, catechins, polyphenols, ellagic acid gingerols, derivatives thereof, and a combination of two or more thereof.
